(19) United States
(12) Patent Application Publication
(10) Pub. No.: US 2013/0095090 A1
(43) Pub. Date: Apr. 18, 2013

(54) FACTOR VIIA COMPLEX USING AN IMMUNOGLOBULIN FRAGMENT

(71) Applicants: Dae Hae Song, Hwaseong-si (KR); Sung In Lim, Hwaseong-si (KR); Chang Hwan Kim, Suwon-si (KR); Sung Kap Hong, Yongin-si (KR); Dae Seong Im, Yongin-si (KR); Se Chang Kwon, Seoul (KR)

(72) Inventors: Dae Hae Song, Hwaseong-si (KR); Sung In Lim, Hwaseong-si (KR); Chang Hwan Kim, Suwon-si (KR); Sung Kap Hong, Yongin-si (KR); Dae Seong Im, Yongin-si (KR); Se Chang Kwon, Seoul (KR)

(73) Assignee: HANMI SCIENCE CO., LTD., Hwaseong-si, Gyeonggi-do (KR)

(21) Appl. No.: 13/807,572

(22) PCT Filed: Jun. 30, 2011
(86) PCT No.: PCT/KR2011/004796

§ 371 Date: Dec. 28, 2012

(30) Foreign Application Priority Data
Jun. 30, 2010 (KR) 10-2010-0062860

(51) Int. Cl. C12N 9/96

(52) U.S. Cl. CPC C12N 9/96
USPC 424/94.2; 435/188

(57) ABSTRACT

Disclosed are a blood coagulation factor complex in which FacVIIa, a non-peptidyl polymer and an immunoglobulin Fc region are bonded by covalent bonds, and the uses thereof. The FacVIIa complex guarantees the in vivo activity of FacVIIa and significantly enhances the serum half life of FacVIIa, so that it is useful for developing long-acting FacVIIa formulations which can improve the compliance of role behavior of patients whose blood does not coagulate.

### TECHNICAL FIELD

The present invention relates to a blood coagulation complex for use in long-acting formulations of factor VIIa (FacVIIa). More particularly, the present invention relates to a blood coagulation complex in which FacVIIa, a non-peptidyl polymer and the immunoglobulin Fc region are held together by covalent bonds, so that the serum half-life is significantly enhanced, the blood clotting function is maintained, and the compliance of the role behavior of patients is dramatically improved. Also, the present invention is concerned with a method for preparing the blood coagulation factor complex.

### BACKGROUND ART

It is estimated that 140,000 people in the world have hemophilia, and that the incidence rate increases by 20% each year. Genetically, hemophilia occurs once in every 10,000 births, but only around 25% of the total cases of hemophilia are diagnosed or treated. One of the greatest problems occurring upon treatment with blood coagulation factors is the development of antibodies against the conventional medicines. Hemophilia A is caused by blood coagulation factor VIII deficiency and is the most common form of the disorder, accounting for 80% of hemophilia cases. Hemophilia B is a disorder involving a lack of blood coagulation factor XI, and comprises approximately 20% of hemophilia cases. Some hemophiliacs develop antibodies against the blood coagulation factors given to them, with a frequency of 10˜15% for hemophilia A and 1˜3% for hemophilia B.

FacVIIa is an active form of FacVII. FacVII, produced by the liver, is an enzyme composed of 406 amino acids, with the gamma-carboxylated glutamic acid at position 10, N-glycosylated asparagines at positions 145 and 322, and O-glycosylated serines at positions 52 and 60. It has two EGF-like domains and one serine protease domain and is activated by the bond cleavage between arginine at position 152 and isoleucine at position 153, which results in the exposure of an active site in a heavy chain. In this process, FacVII which is a single chain bonded to a light chain and heavy chain together is converted to FacVIIa which has a two-chain structure with separated light and heavy chains.

Unlike other various blood coagulation factors, FacVIIa acts in the subsidiary pathway of the blood coagulation mechanism, without producing antibodies, so that it can be administered at a high dose. Thus, FacVIIa is applicable for the treatment of both hemophilia A and B, and starts to be used as an alternative to conventional therapeutics for hemophilia due to its ability to be used safely in terms of antibody production and high dose administration. Also, the production of antibodies in response to the factors given to hemophiliacs makes it difficult to apply other coagulation factors the FacVIIa.

However, FacVIIa, although not causing the production of antibodies thereto, has the shortest serum half-life among the various blood coagulation factors. Thus, FacVIIa has to be administered not only frequently, causing pain to patients, but also in a large amount, imparting an economic burden to patients. To overcome these disadvantages, FacVIIa should be formulated into a long-acting form anticipated to be used for the prophylaxis of hemophilia, but not only as a supplemental factor upon hemorrhage.

rVIIa-FP (CSL Behring) in which albumin is fused to the C-terminus of FacVIIa is in the pre-clinical phase and was found to have a serum half-life that was a 6.7-fold increase compared to native FacVIIa in rats. However, a serum half-life as short of 4.38 hours is very short so that it is still insufficient for it to be effectively used in the prophylaxis and treatment of hemophilia.

Also, PEGLip-FVIIa (Omri), a pegylated liposome formulation of FacVIIa, is in the pre-clinical phase and has a serum half-life only twice as long as that of native FacVIIa.

MAXY-VII (Bayer/Maxygen) and NN7128 (Novo/Neose) are Factor VII products both of which have prolonged serum half lives by Gla domain mutation and hyperglycosylation and by 40K PEGylation, respectively, but in the progress of phase 1 and 2 study for each their serum half-life is five times longer than that of native FacVII, which is still insufficient for the prophylaxis and treatment of hemophilia.

### DISCLOSURE

### Technical Problem

Leading to the present invention, as a way to maximize the enhancement of serum half-life and the maintenance of in vivo activity at the same time, the preparation method was used wherein immunoglobulin Fc, non-peptidyl polymer and FacVIIa were site-specifically connected to each other via covalent bonds. As a result, a serum half-life of blood coagulation factor complex is dramatically prolonged as long as 60 hrs and confirmed to be much longer than that obtained by conventional pegylation or in-frame fusion techniques.

### Technical Solution

It is therefore an object of the present invention to provide a FacVIIa complex which allows the serum half-life of FacVIIa to be prolonged with the maintenance of in vivo activity in a relatively high level, thus exerting excellent coagulation functionality, a long-acting formulation comprising the same, and a method for the preparation thereof.

### Advantageous Effects

As described hitherto, the FacVIIa complex of the present invention guarantees the in vivo activity of FacVIIa and significantly enhances the serum half live of FacVIIa, so that it is useful for developing long-acting FacVIIa formulations which can the compliance of role behaviors of patients whose blood does not coagulate.

### DESCRIPTION OF DRAWINGS

FIG. 1 is a plot showing changes in the blood levels of FacVIIa and immunoglobulin Fc-PEG-FacVIIa with time in SD rats.

FIG. 2 is a graph showing results of a comparative in vitro efficacy test of Novoseven, FacVIIa, and immunoglobulin Fc-PEG-FacVIIa.

FIG. 3 is a graph showing results of a comparative in vivo efficacy test of Novoseven, FacVIIa, and immunoglobulin Fc-PEG-FacVIIa.

### BEST MODE

In accordance with an aspect thereof for accomplishing the above object, the present invention addresses a FacVIIa complex in which FacVIIa is linked to an immunoglobulin Fc region via a non-peptidyl polymer.

As used herein the term “FacVIIa” refers to an active form of coagulation factor VII.

Preferably, the FacVIIa complex of the present invention is prepared through the activation of a FacVII complex. In this context, a long-acting complex is prepared from FacVII, a non-peptidyl polymer and an immunoglobulin Fc region and then allowed to undergo an activation process to form a FacVIIa complex comprised of FacVIIa, a non-peptidyl polymer and an immunoglobulin Fc region, during which in vivo activity of the complex is increased and it becomes structurally more homogeneous.

The activation process of converting the FacVII complex into the FacVIIa complex may include, but is not limited to, an on-column activation process and an in-solution activation process. With a preference, an on-column activation was used for FacVII complex in this invention.

In an on-column activation process, also called, solid-phase activation process, a FacVII complex is immobilized onto an anionic exchange column and then subjected to “autoactivation” without particular additives.

Unlike the on-column activation process, the in-solution activation process requires various factors, for example, calcium ion concentration, pH, temperature and FacVII concentration, for the activation of FacVII.

The FacVIIa of the present invention is an activated peptide of FacVII that is involved in the subsidiary pathway of the blood coagulation mechanism. Among the peptide are an active form of native FacVII, FacVIIa agonists, precursors, derivatives, fragments, and variants.

The term “FacVIIa agonist,” as used herein, refers to a substance that exhibits the same biological activity as that of FacVIIa irrespective of the structure of FacVIIa.

The term “FacVIIa derivative,” as used herein, refers to a peptide that has the function of regulating blood coagulation in vivo, with at least 80% amino acid sequence homology to native FacVIIa and that may be modified at some amino acid residues by chemical substitution (e.g., alpha-methylation, alpha-hydroxylation), deletion (e.g., deamination) or decoration (e.g., N-methylation).

The term “FacVIIa derivative,” as used herein, refers to a peptide in which one or more amino acid residues are added to or deleted from the amino acid sequence of FacVIIa and that has blood coagulation activity in vivo. The added amino acid residues may be non-natural amino acids (e.g., D-amino acid).

The term “FacVIIa variant,” as used herein, refers to a peptide that is different in amino acid sequence by one or more amino acids from FacVIIa and that has blood coagulation activity in vivo.

In addition to individual FacVIIa agonists, derivatives, fragments and variants, a peptide having a combination of the properties thereof, for example, a peptide that is different in amino acid sequence by one or more amino acid residues and is deaminated at its N-terminal amino acid may be used so long as it has a blood clotting function.

Preferably, the FacVIIa complex of the present invention is composed of a non-peptidyl polymer, an immunoglobulin Fc region and FacVIIa, with a linkage between one end of the non-peptidyl polymer and the immunoglobulin Fc region and between the other end of the non-peptidyl polymer and the N-terminus of FacVIIa.

More preferably, in the FacVIIa complex of the present invention, the non-peptidyl is linked at one end to the immunoglobulin Fc region and at the other end to the N-terminus of the light chain of FacVIIa.

The term “N-terminus”, used in the context of FacVIIa, is intended to encompass a region containing the N-terminus of FacVIIa. In the FacVIIa complex of the present invention, therefore, the non-peptidyl polymer may be linked to the very N-terminal amino acid residue of FacVIIa or to an amino acid residue somewhat distant from the N-terminus so long as the FacVIIa complex retains the desired function.

Since FacVII is a single chain structure in which a light chain and a heavy chain are linked to each other before activation, only the N-terminus of the light chain is exposed outside. Converting FacVII to FacVIIa, cleavage between the arginine at position 152 and the isoleucine at position 153, exposes the active site of the heavy chain, with the isoleucine at position 153 accounting for the N-terminus of the heavy chain. Because the N-terminus of the heavy chain plays an important role in the activity of FacVIIa, the polymer must be linked to the N-terminus of the light chain, but not the heavy chain, so as to increase the titer.

In an embodiment, PEG is linked to the N-terminus of an immunoglobulin Fc region and selectively coupled to the N-terminus of the light chain of FacVII to give a FacVII-PEG-immunoglobulin Fc complex. Afterwards, an additional activation process is carried out to complete the FacVIIa-PEG-immunoglobulin Fc complex. The FacVIIa-PEG-immunoglobulin Fc complex prepared according to the present invention has a serum half-life of 60 hours, much longer than that of conventional therapeutic agents, and exhibits excellent blood coagulation effects in animal models, so that it can be prepared into long-acting FacVIIa formulations that retain excellent in vivo activity.

An immunoglobulin Fc region is a biodegradable polypeptide which can be metabolized in vivo, so that it can safely be used as a drug carrier. In addition, an immunoglobulin Fc region is more advantageous in terms of production, purification and production yield because of its relatively smaller size compared to an entire immunoglobulin molecule. In addition, because amino acid sequence differs in one antibody to another, it can be expected that removal of highly heterogeneous Fab greatly increases homogeneity of substance and lower the likelihood of inducing blood antigenicity.

As used herein, the term “immunoglobulin Fc region” refers to an immunoglobulin fragment that is devoid of the variable regions of light and heavy chains, the constant region 1 of the heavy chain (CH1) and the constant region 1 of the light chain (CL1), that is, a fragment comprised of the constant regions 2 and 3 of the heavy chain (CH2 and CH3). Optionally, the constant region of heavy chain may further comprise a hinge region. Also, the immunoglobulin Fc region of the present invention may be an extended Fc region which comprises a part of or the entirety of the constant region 1 of the heavy chain (CH1) and/or the constant region 1 of the light chain (CL1) in addition to the constant regions 2 and 3 of the heavy chain (CH2 and CH3) so long as it shows effects substantially identical or superior to those of the classical Fc region excluding only the variable regions of light and heavy chains of immunoglobulin. Further, it may be the region in which a considerably long part of amino acid sequence corresponding to CH2 and/or CH3 is deleted. Consequently, the immunoglobulin Fc region of the present invention may be composed of 1) CH1 domain, CH2 domain, CH3 domain and CH4 domain, 2) CH1 domain and CH2 domain, 3) CH1 domain and CH3 domain, 4) CH2 domain and CH3 domain, 5) a combination of one or more domains and an immunoglobulin hinge region (or a part of hinge region), or 6) a dimer of each constant domain of the heavy chain and the constant region of the light chain

Further, the immunoglobulin Fc region of the present invention may include not only the wild-type Fc but its amino acid sequence mutant. The term “amino acid sequence mutant”, as used herein, refers to an amino acid sequence that is different from the wild-type as a result of deletion, insertion, conserved or non-conserved substitution of one or more amino acid residues, or a combination thereof. For instance, amino acid residues at positions 214 to 238, 297 to 299, 318 to 322, or 327 to 331 in IgG Fc, known to be important for linkage, may be used as the sites suitable for modification. Various derivatives, such as those prepared by removing the sites of disulfide bonds, removing several N-terminal amino acids from native Fc, or adding methionine to the N-terminus of native Fc, may be available. In addition, complement fixation sites, e.g., C1q fixation sites, or ADCC sites may be eliminated to remove the effector function from the native Fc region. The techniques of preparing amino acid sequence mutants of the immunoglobulin Fc region are disclosed in International Patent Publication Nos. WO 97/34631 and WO 96/32478 and so forth.

Amino acid substitutions in a protein or peptide molecule that do not alter the activity of the molecule are well known in the art (H. Neurath, R. L. Hill, The Proteins, Academic Press, New York, 1979). The most common substitutions occur between amino acid residues Ala/Ser, Val/Ile, Asp/Glu, Thr/Ser, Ala/Gly, Ala/Thr, Ser/Asn, Ala/Val, Ser/Gly, Thr/Phe, Ala/Pro, Lys/Arg, Asp/Asn, Leu/Ile, Leu/Val, Ala/Glu, and Asp/Gly.

*47

Optionally, amino acids may be modified by phosphorylation, sulfation, acrylation, glycosylation, methylation, farnesylation, acetylation, and amidation.

The above-described Fc derivatives exhibit the same biological activity as that of the wild-type, but have improved structural stability when subjected to heat and pH.

These Fc regions may be obtained as native Fc regions from humans or animals such as cow, goats, pigs, mice, rabbits, hamsters, rats, guinea pigs, etc., or as recombinant or derived Fc regions from transformed animal cells or microorganisms. Native Fc regions may be obtained by protease digestion of the gamut of immunoglobulins isolated from human or animal samples. Immunoglobulins are cleaved into Fab and Fc by papain and into pF′ c and F(ab′)2 by pepsin, followed by size-exclusion chromatography to separate Fc or pF′c therefrom.

Preferably, a recombinant human Fc region obtained from a microorganism is preferred.

The immunoglobulin Fc region useful in the present invention may be glycosylated to the same extent as or to a higher and lesser extent than the native form or may be deglycosylated. Increased or decreased glycosylation or deglycosylation of the immunoglobulin region may be achieved by typical methods, for example, by using a chemical method, an enzymatic method or a genetic engineering method using a microorganism. Herein, when deglycosylated, the complement (C1q) binding of an immunoglobulin Fc region becomes significantly decreased and it has reduced or no antibody-dependent cytotoxicity or complement-dependent cytotoxicity, so that it does not induce unnecessary immune responses in vivo. In this context, deglycosylated or aglycosylated immunoglobulin Fc regions are more consistent with the purpose of being as drug carriers.

The term “deglycosylation”, as used herein, is intended to mean the enzymatic removal of sugars from an Fc region. The term “aglycosylation”, when used in conjunction with an Fc region, means an Fc region free of sugars, expressed from prokaryotes, preferably from E. coli.

The immunoglobulin Fc region may originate from humans or animals such as cows, goats, pigs, mice, rabbits, hamsters, rats, guinea pigs, etc., and preferably is of human origin. In addition, the immunoglobulin Fc region may be derived from IgG, IgA, IgD, IgE, IgM, or combinations or hybrids thereof. Preferably, the Fc region is derived from IgG or IgM, which are the most abundant ones in human blood, and most preferably from IgG, which is known to improve the serum half-life of ligand-binding proteins.

The “combination”, as used herein, means that polypeptides encoding single-chain immunoglobulin Fc regions of the same origin are linked to a single-chain polypeptide of a different origin to form a dimer or multimer. That is, a dimer or multimer may be formed from two or more fragments selected from the group consisting of IgG1 Fc, IgG2 Fc, IgG3 Fc and IgG4 Fc fragments.

The term “hybrid”, as used herein, means that sequences encoding two or more immunoglobulin Fc fragments of different origin are present in a single-chain immunoglobulin Fc fragment. In the present invention, various types of hybrids are possible. That is, domain hybrids may be composed of one to four domains selected from the group consisting of CH1, CH2, CH3 and CH4 of IgG Fc, IgM Fc, IgA Fc, IgE Fc and IgD Fc, and may include the hinge region.

IgG is divided into the IgG1, IgG2, IgG3 and IgG4 subclasses, and the present invention may include combinations or hybrids thereof. Preferred are the IgG2 and IgG4 subclasses, and most preferred is the Fc region of IgG4 rarely having effector functions such as CDC (Complement Dependent Cytotoxicity).

That is, the immunoglobulin Fc region most suitable as the drug carrier of the present invention is a human IgG4-derived aglycosylated Fc region. The human-derived Fc region is more preferable than a non-human derived Fc region, which may act as an antigen in the human body and cause undesirable immune responses such as the production of a new antibody against the antigen.

The term “non-peptidyl polymer”, as used herein, refers to a biocompatible polymer comprised of at least two repeating units which are held together by any covalent bond other than a peptide bond. The non-peptidyl polymer may have two or three terminal functional groups.

For this invention, a useful non-peptidyl polymer may be selected from polyethylene glycol, poly propylene glycol, copolymers of ethylene glycol and propylene glycol, polyoxyethylated polyols, polyvinyl alcohol, polysaccharides, dextran, polyvinyl ethyl ether, biodegradable polymers such as PLA (poly(lactic acid) and PLGA (poly (lactic-glycolic acid), lipid polymers, chitins, hyaluronic acid, and a combination thereof. The most preferred is polyethylene glycol. Their derivatives are well known in the art and derivatives which can be readily prepared using a method known in the art are also within the scope of the present invention.

Conventional peptidyl polymers used in fusion proteins constructed by an in-frame fusion technique are disadvantageous in that they are readily cleaved in vivo by proteinases and thus cannot guarantee the prolongation of serum half-life by the carrier. contrast, the polymer of the present invention, resistant against proteinases, maintains the serum half-life of the peptides, like the carriers. Therefore, as long as it is resistant to in vivo proteineases, any non-peptidyl polymer may be used in the present invention, without limitation. The non-peptidyl polymer ranges in molecular weight from 1 to 100 kDa and preferably from 1 to 20 kDa. in addition, the non-peptidyl polymer which is linked to the immunoglobulin Fc region may be not only an individual polymer but a combination of different polymers.

The non-peptidyl polymer useful in the present invention has functional groups which are coupled to an immunoglobulin Fc region and a protein drug.

The non-peptidyl polymer discussed above has two or three termini, And the functional group is preferably selected from the group consisting of aldehyde, propion aldehyde, butyl aldehyde, maleimide, and succinimide derivative. As for succinimide, its derivatives including, succinimidyl propionate, hydroxy succinimidyl, succinimidyl carboxymethyl or succinimidyl carbonate may also be used. Particularly when the non-peptidyl polymer has aldehyde functional groups at its both ends, it can be effectively linked at both ends to a physiologically active polypeptide and an immunoglobulin, respectively, with minimal non-specific reactions therebetween. The final products produced by reductive alkylation via an aldehyde bond are much more stable than those linked via an amide bond. An aldehyde functional group specifically reacts with an amino terminus at low pH, and can form a covalent bond with a lysine residue at high pH, e.g., a pH of 9.0.

The two or three terminal functional groups of the non-peptidyl polymer may be the same or different. For example, the non-peptidyl polymer may have a maleimide group at one end and an aldehyde group, a propionaldehyde group, or a butyl aldehyde group at the other or another end. When poly(ethylene glycol) with an hydroxy group at both ends is used as a non-peptidyl polymer, the hydroxy group may be activated into the above-mentioned functional groups before being used in the present invention. Alternatively, commercially available poly(ethylene glycol) with modified functional groups may be used to prepare the protein complex of the present invention.

In accordance with another aspect thereof, the present invention provides a pharmaceutical composition for blood coagulation, comprising the FacVIIa complex.

Preferably, the present invention provides a pharmaceutical composition for the treatment of blood coagulation-related diseases including hemophilia, bleeding, acute intracerebral hemorrhage, wounds and FacVII deficiency.

The term “administration”, as used herein, means the introduction of a predetermined amount of a substance into a patient by a certain suitable method. So long as it is able to induce the complex to reach a target tissue, any route of administration may be used. A variety of modes of administration are contemplated, including intraperitoneally, intravenously, intramuscularly, subcutaneously, intradermally, orally, topically, intranasally, intrapulmonarily and intrarectally, but the present invention is not limited to these modes of administration. However, since peptides are digested upon oral administration, active ingredients of the composition for oral administration should be coated or formulated for protection against degradation in the stomach. Preferably, the composition of the present invention may be administered in an injectable form. In addition, the pharmaceutical composition of the present invention may be administered using a certain apparatus capable of transporting the active ingredients into a target cell.

The pharmaceutical composition comprising the complex according to the present invention may comprise a pharmaceutically acceptable carrier. For oral administration, the pharmaceutically acceptable carrier may include binders, lubricants, disintegrators, excipients, solubilizers, dispersing agents, stabilizers, suspending agents, coloring agents and odoriferous substances. For injectable preparations, the pharmaceutically acceptable carrier may include buffering agents, preserving agents, analgesics, solubilizers, isotonic agents and stabilizers. For preparations for topical administration, the pharmaceutically acceptable carrier may include bases, excipients, lubricants and preserving agents. The pharmaceutical composition of the present invention may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into a unit dosage form, such as an ampule in single-dose dosage form or a multidose container. The pharmaceutical composition may also be formulated into solutions, suspensions, tablets, pills, capsules and long-acting preparations.

Examples of carriers, excipients and diluents suitable for the pharmaceutical formulations include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, odoriferous substances, and antiseptics.

A dosage of the pharmaceutical composition of the present invention may be determined by the type of the drug which is the active component as well as by several related factors including the type of disease to be treated, administration route, the patient's age, gender, weight and severity of the illness. Since the pharmaceutical composition of the present invention has a very long duration of action in vivo and a high titer, it has the advantage of greatly reducing the frequency of administration of the pharmaceutical drugs.

In accordance with a further aspect thereof, the present invention provides a method for treating a blood coagulation-related disease, which comprises administering the FacVIIa complex or the pharmaceutical composition of this invention to a subject in needs thereof.

Preferably, the disease is one caused by the insufficient coagulation of blood, and may include, but is not limited to, hemophilia, bleeding, acute intracerebral hemorrhage, wounds, and FacVII deficiency.

The subject may be mammals including, but not limited to, humans, mice, pigs, cows, dogs, sheep, etc. with a preference for humans.

The FacVIIa complex, the composition and the administration are as described above.

In accordance with still a further aspect thereof, the present invention provides a method for preparing a FacVIIa complex, comprising:

(1) linking a non-peptidyl polymer having an aldehyde, a maleimide or a succinimide derivative as a terminal functional group to an amine group of an immunoglobulin Fc region via a covalent bond to give a conjugate;

(2) isolating the non-peptidyl polymer-immunoglobulin Fc region conjugate from the reaction mixture of step (1);

(3) covalently linking FacVII to another end of the non-peptidyl polymer of the isolated conjugate to afford a FacVII complex in which the non-peptidyl polymer is linked at one end to the immunoglobulin Fc region and at another end to FacVII; and

(4) activating the FacVII complex of step (3) into a FacVIIa complex in which FacVIIa is linked to the immunoglobulin Fc region via the non-peptidyl polymer.

In accordance with still another aspect thereof, the present invention provides a method for preparing a FacVIIa complex, comprising:

(1) linking a non-peptidyl polymer having an aldehyde group at each terminus to the N-terminus of an immunoglobulin Fc via a covalent bond at a pH of 5.0 to 7.0 to give an immunoglobulin-non-peptidyl polymer conjugate;

(2) isolating the conjugate from the reaction mixture of step (1);

(3) covalently linking FacVII to another end of the non-peptidyl polymer of the conjugate to form a FacVII complex in which the non-peptidyl polymer is linked at one end to the immunoglobulin Fc region and at another end to FacVII; and

(4) activating the FacVII complex of step (3) into a FacVIIa complex in which FacVIIa is linked to the immunoglobulin Fc region via the non-peptidyl polymer.

In accordance with still another aspect thereof, the present invention provides a method for preparing a FacVIIa complex, comprising:

(1) linking a non-peptidyl polymer having an aldehyde group at each terminus to FacVII via a covalent bond to give a conjugate;

(2) isolating the FacVII-non-peptidyl polymer conjugate from the reaction mixture of step (1);

(3) covalently linking an immunoglobulin Fc region to another end of the non-peptidyl polymer of the isolated conjugate to afford a FacVII complex in which the non-peptidyl polymer is linked at one end to the immunoglobulin Fc region and at another end to FacVII; and

(4) activating the FacVII complex of step (3) into a FacVIIa complex in which FacVIIa is linked to the immunoglobulin Fc region via the non-peptidyl polymer.

Preferably, the FacVII complex is attached to an anion exchange column and activated into the FacVIIa complex by on-column activation (autoactivation).

In the method, the FacVII is linked preferably at its N-terminus to the non-peptidyl polymer.

More preferably, the N-terminus mentioned above is from the light chain of FacVII.

In a preferred embodiment of the method, FacVII is native FacVII, or a FacVII agonist, precursor, derivative, fragment or variant. Most preferred is the native FacVII.

In another preferred embodiment of the method, the FacVIIa is native FacVIIa, or a FacVIIa agonist, precursor, derivative, fragment or variant. Most preferred is native FacVIIa.

Examples of the non-peptidyl polymer useful in the method of the present invention include polyethylene glycol, polypropylene glycol, copolymers of ethylene glycol and propylene glycol, polyoxyethylated polyols, polyvinyl alcohol, polysaccharides, dextran, polyvinyl ethyl ether, biodegradable polymers such as PLA (poly(lactic acid)) and PLGA (poly(lactic-glycolic acid), lipid polymers, chitins, and hyaluronic acid. The most preferred is polyethylene glycol.

In the method of the present invention, preferably, the non-peptidyl polymer has an aldehyde derivative as a terminal group and more preferably has aldehyde functional groups at three termini.

### MODE FOR INVENTION

A better understanding of the present invention may be obtained through the following examples which are set forth to illustrate, but are not to be construed as limiting the present invention.

### Example 1

### Preparation of Immunoglobulin Fc-PEG-FacVIIa Complex

An immunoglobulin Fc was pegylated at the N terminus with 5K PropionALD(3) PEG (PEG with three terminal propionaldehyde groups, NOF, Japan). In this regard, 6 mg/mL immunoglobulin Fc was reacted with PEG at 4° C. for 4.5 hrs with the molar ratio of immunoglobulin Fc to PEG set at 1:2. The reaction was performed in 100 mM potassium phosphate buffer at pH 6.0 in the presence of 20 mM SCB (NaCNBH₃) as a reducing agent. The reaction mixture was loaded onto SOURCE Q (LRC25 85 ml, Pall Corporation) to purify the mono-pegylated immunoglobulin Fc. Thereafter, FVII was coupled with the immunoglobulin Fc-5K PEG at a molar ratio of 1:10 (FVII immunoglobulin Fc-5K PEG) at 4° C. for 18 hrs, with the total protein concentration set to 20 mg/mL. The coupling reaction was performed in 100 mM potassium phosphate at pH 6.0 in the presence of 20 mM SCB as a reducing agent. The coupling reaction mixture was purified by passing it through two columns. To remove the immunoglobulin Fc-5K PEG conjugate which remained uncoupled, SOURCE Q (LRC25 85 ml, Pall Corporation) was employed. Given a salt gradient of 1M NaCl in 20 mM Tris (pH 7.5), the column eluted immunoglobulin Fc-5K first due to relatively weak linkage and then immunoglobulin Fc-3 armPEG-FVII. Thereafter, secondary purification was carried out using a SOURCE ISO (GE Healthcare) column to isolate immunoglobulin Fc-3 armPEG-FVII from FVII and FVII multimer impurities. In this context, FVII, immunoglobulin Fc-3 arm PEG-FVII, and FVII multimer impurities were eluted in that order.

To be activated, the immunoglobulin Fc-3 arm PEG-FVII was reloaded onto SOURCE Q, followed by pouring a mobile phase containing 1.75 mM calcium ion on the column for 6 hours. Elution was carried out with 35 mM calcium ions to afford immunoglobulin Fc-3 arm PEG-FVIIa.

Column: Source Q (LRC25 85 ml, Pall Corporation)

Flow rate: 4 ml/min

Gradient: A 0→7% 1 min B, 7%→40% 80 min B (A: 20 mM Tris pH7.5, B: A+1M NaCl)

Column: SOURCE ISO (23 ml, 16/10 HR column, GE Healthcare)

Flow rate: 2 ml/min

Gradient: B 100→40% 60 min A (A: 20 mM Tris pH7.5, B: A+1.6M (NH₄)₂SO₄)

Column: Source Q (15 ml, 16/10 HR column, GE Healthcare)

Flow rate: 1 ml/min

Mobile phase: 20 mM Tris pH7.5+1.75 mM CaCl₂ 1.25 mM NaCl

### Example 2

### Preparation of 20 k PEG-FacVIIa(N) Conjugate

FVII (FacVII) was pegylated at the N terminus with 20K mPEG butylaldehyde (Nektar, USA). In this regard, 5 mg/ml, FVII was reacted with PEG at 4° C. for 10 hrs with the molar ratio of FVII to 20K PEG set at 1:3. The reaction was performed in 100 mM sodium acetate buffer at pH 5.0 in the presence of 20 mM SCB (NaCNBH₃) as a reducing agent. The mono-pegylated FVII was purified through RESOURCE Q (1 ml, prepacked, GE Healthcare). Given a salt gradient of 1M NaCl in 20 mM Tris (pH 7.5), the column eluted multi-pegylated FVII, mono-pegylated FVII and FVII in that order. Thereafter, secondary purification was carried out using a Superdex_{—}200 (Hiroad 16/60, GE Healthcare) Column to isolate mono-pegylated FVII from FVII and FVII multimer impurities. To be activated, the mono-pegylated FVII was reloaded onto SOURCE Q, followed by pouring a mobile phase containing 1.75 mM calcium ion on the column for 1 hour. Elution was carried out with 35 mM calcium ions to afford mono-pegylated FVIIa.

Column: RESOURCE Q (1 ml, prepacked, GE Healthcare)

Flow rate: 0.5 ml/min

Gradient: A 0→50% 50 min B (A: 20 mM Tris pH7.5, B: A+1M NaCl)

Column: Superdex_{—}200 (Hiroad 16/60 HR column, GE Healthcare)

Flow rate: 1 ml/min

Mobile phase: PBS

Column: RESOURCE Q (1 ml, prepacked, GE Healthcare)

Flow rate: 0.5 ml/min

Mobile phase: 20 mM Tris pH7.5+1.75 mM CaCl₂ 1.25 mM NaCl

### Example 3

### Preparation of 20K PEG-FacVIIa(Lys) Conjugate

FVII was pegylated at a lysine residue with 20 k mPEG SPA (Nektar, USA). In this regard, 3 mg/mL FVII was reacted with 20 k PEG at room temperature for 3 hrs with the molar ratio of FVII to 20 k PEG set at 1:5. The reaction was performed in 100 mM sodium phosphate buffer at pH 8.0. The mono-pegylated FVII was purified through RESOURCE Q (1 ml, prepacked, GE Healthcare). Given a salt gradient of 1M NaCl in 20 mM Tris (pH 7.5), the column eluted multi-pegylated FVII, mono-pegylated FVII and FVII in that order. Then, secondary purification was carried out on a Superdex_{—}200 (Hiroad 16/60, GE Healthcare) Column to isolate mono-pegylated FVII from FVII and FVII multimer impurities. To be activated, the purified mono-pegylated FVII was reloaded onto SOURCE Q, followed by pouring a mobile phase containing 1.75 mM calcium ion on the column. for 1 hour. Elution was carried out with 35 mM calcium ions to afford mono-pegylated FVIIa.

Column: SOURCE Q (23 ml, HR Column, GE Healthcare)

Flow Rate: 2 ml/min

Gradient: A 0→50% 50 min B (A: 20 mM Tris pH7.5, B: A+1M NaCl)

Column: Superdex_{—}200 (Hiroad 16/60 HR column, GE Healthcare)

Flow Rate: 1 ml/min

Mobile Phase: PBS

Column: RESOURCE Q (1 ml, prepacked, GE Healthcare)

Flow Rate: 0.5 ml/min

Mobile Phase: 20 mM Tris pH7.5+1.75 mM CaCl₂ 1.25 mM NaCl

### Example 4

### Measurement of Serum Half-Life of FVIIa and Immunoglobulin Fc-PEG-FVIIa

To evaluate the pharmacokinetic parameters thereof, FVIIa and immunoglobulin Fc-PEG-FVIIa were each intravenously injected at a dose of 100 μg/kg into normal SD rats, followed by ELISA analysis to obtain serum levels.

Following intravenous injection, 0.5 mL of a blood sample was collected at 0.25, 0.5, 1, 2, 5, 10, 24, and 48 hrs for the FVIIa-administered rats, and at 0.25, 0.5, 1, 2, 5, 10, 24, 48, 72, 96 and 120 hrs for the immunoglobulin Fc-PEG-FVIIa-administered rats. The blood samples were collected in tubes with sodium citrate to prevent coagulation, and centrifuged for 5 min using an Eppendorf high-speed micro centrifugator to separate serum. Serum protein levels were measured by ELISA (IMUBIND, Factor VIIa ELISA Kit, American diagnostic inc.) using antibodies specific to FVIIa.

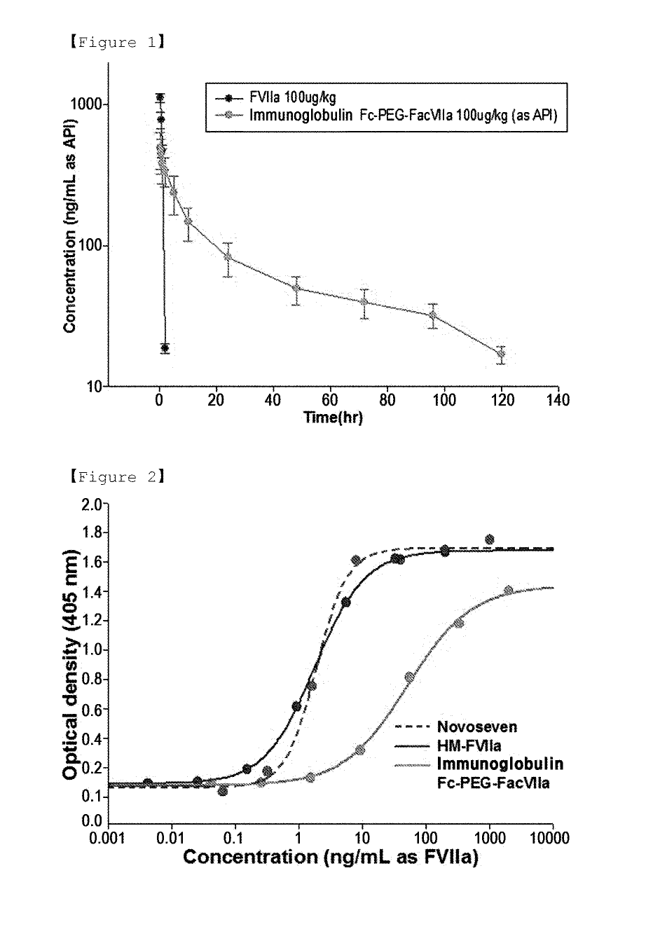

The serum concentration-time curve and the results of pharmacokinetic analyses of FVIIa and immunoglobulin Fc-PEG-FVIIa are given in FIG. 1 and Table 1. In the following table, Tmax accounts for the time taken to reach the maximal serum concentration of a drug, T1/2 for the serum half-life of a drug, and MRT (mean residence time) for the mean time during which a drug molecule resides in the body.

As shown in Table 1 and FIG. 1, immunoglobulin Fc-PEG-FVIIa was observed to have a great serum half-life of as long as about 60 hrs.

TABLE 1
Pharmacokinetics of FVIIa and immunoglobulin Fc-PEG-FVIIa in SD Rats

| | | | Immunoglobulin |
|---|---|---|---|
| | | FVIIa | Fc-PEG-FVIIa |
| Tmax | (hr) | 0.25 | 0.25 |
| T1/2 | (hr) | 0.284 | 60.4 |
| MRT | (hr) | 0.63 | 32.71 |

### Example 5

### Measurement of In Vitro Activity of FVIIa and Immunoglobulin Fc-PEG-FVIIa

To determine in vitro activity of native FVIIa and the immunoglobulin Fc-PEG-FVIIa prepared in Example 1, a chromogenic assay was carried out with a commercially available kit (Chromogenix, COASET). As a control, Novoseven was used which is a recombinant form of FVIIa commercially available from Novo Nordisk, which is applied to the treatment for bleeding of hemophiliac and the hemostasis of patients under a surgical operation.

Activity assay was performed according to the instructions described in “2.7.10. ASSAY OF HUMAN COAGULATION FACTOR VII” of the European Pharmacopoeia. FX was activated into FXa by treatment with dilutions of Novoseven, FVIIa and immunoglobulin Fc-PEG-FVIIa at various concentrations and S-2765 used as a substrate was hydrolyzed into a peptide and pNA, a chromophoric group, by the FXa. The yellow color of the hydrolyzed pNA was used to measure absorbance at 405 nm on an ELIAS reader. A dose responsive curve and EC50 values were determined using the measured absorbance and the treated concentrations of the drug. As a result, immunoglobulin Fc-PEG-FacVIIa was observed to have an EC50 of 50.72 ng/mL, which is 27-fold higher than that of Novoseven [FIG. 2].

TABLE 2
EC50 and Specific Activity of Novoseven, FVIIa, and
Immunoglobulin Fc-PEG-FVIIa

| | Lot. No. | EC50 (as FVIIa) |
|---|---|---|
| Novoseven | PU60399 | 1.87 ng/mL |
| FVIIa | B13160-PJE271 | 1.77 ng/mL |
| immunoglobulinFc-PEG- | B13160-LJE131 | 50.72 ng/mL |
| FacVIIa | | |

### Example 6

### Measurement of In Vivo Activity of FVIIa and Immunoglobulin Fc-PEG-FVIIa

FVIIa and immunoglobulin Fc-PEG-FVIIa were assayed for in vivo FacVIIa activity depending on the administration of test drugs in SD rats pre-treated with warfarin. As a control, Novoseven was used which is a recombinant form of FVIIa commercially available from Novo Nordisk, which is applied to the treatment for bleeding hemophilias and the hemostasis of patients under a surgical operation.

Warfarin, which acts to inhibit the gamma-carboxylation of vitamin K-dependent coagulation factors such as Factor II, IX, X and VII, was administered to SD rats 24 hrs ahead, after which Novoseven, FVIIa, and immunoglobulin Fc-PEG-FVIIa were individually intravenously injected at dosages of 250 μg to the SD rats. One mL of blood was sampled from the jugular vein at 0.4, 4, 24, and 48 hrs after the intravenous injection, using tubes containing sodium citrate. FVII activity (%) from the isolated serum was measured using ACL9000 (Werfen group).

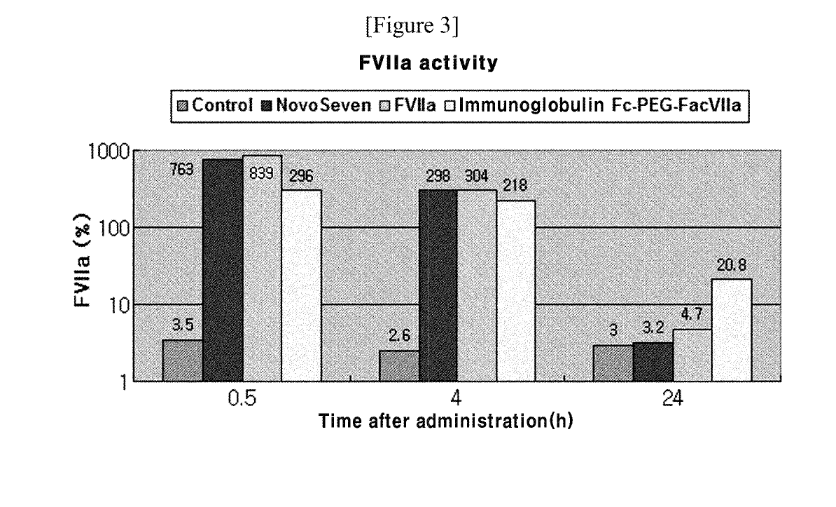

As a result, similar in vivo activities were observed for FVIIa and Novoseven. For the immunoglobulin Fc-PEG-FVIIa, its activity was lower at 25 min and 4 hrs after administration, compared to Novoseven, but stayed 6.5-fold higher at 24 hrs after administration, compared to Novoseven (Table 3, FIG. 3).

TABLE 3
In Vivo Activity of Novoseven, FVIIa, and Immunoglobulin Fc-PEG-FVIIa
with Time

| | | FVII (%) | | | |
|---|---|---|---|---|---|
| Group | | 25 min | 4 hr | 24 hr | 48 hr |
| Non-treat | Vehicle | 218.8 ± 39.1 | 183.6 ± 9.4 | 240.8 ± 40.1 | 239.4 ± 24.6 |
| Warfarin | Vehicle | 3.5 ± 1.5 | 2.6 ± 0.7 | 3.0 ± 0.8 | 16.9 ± 11.5 |
| Pre-treatment | Novoseven | 762.6 ± 138.1 | 298.2 ± 169.1 | 3.2 ± 0.8 | 28.7 ± 26.0 |
| 10 mg/kg | FVIIa | 838.8 ± 147.9 | 303.8 ± 59.2 | 4.7 ± 3.6 | 39.5 ± 44.2 |
| | Ig | 295.8 ± 51.3 | 217.6 ± 34.1 | 20.8 ± 5.3 | 27.9 ± 24.0 |
| | Fc-PEG-FacVIIa | | | | |

As described hitherto, the FacVIIa complex of the present invention guarantees the in vivo activity of FacVIIa and significantly enhances the serum half live of FacVIIa, so that it is useful for developing long-acting FacVIIa formulations which can be in compliance with the role behaviors of patients whose blood do not coagulate.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A FacVIIa complex, comprising FacVIIa linked to an immunoglobulin Fc region via a non-peptidyl polymer, said non-peptidyl polymer being selected from the group consisting of polyethylene glycol, polypropylene glycol, copolymers of ethylene glycol and propylene glycol, polyoxyethylated polyols, polyvinyl alcohol, polysaccharides, dextran, polyvinyl ethyl ether, biodegradable polymers, lipid polymers, chitins, hyaluronic acid, and a combination thereof.

2. The FacVIIa complex of claim 1, wherein the non-peptidyl polymer is linked at each terminus to the immunoglobulin Fc region and an N-terminus of FacVIIa.

3. The FacVIIa complex of claim 1, wherein the non-peptidyl polymer is linked at each terminus to the immunoglobulin Fc region and an N-terminus of a light chain of FacVIIa.

4. The FacVIIa complex of claim 1, wherein the immunoglobulin Fc region is aglycosylated.

5. The FacVIIa complex of claim 1, wherein the immunoglobulin Fc region comprises one to four domains selected from the group consisting of CH1, CH2, CH3 and CH4 domains.

6. The FacVIIa complex of claim 5, wherein the immunoglobulin Fc region further comprises a hinge region.

7. The FacVIIa complex of claim 1, wherein the immunoglobulin Fc region is derived from IgG, IgA, IgD, IgE or IgM.

8. The FacVIIa complex of claim 7, wherein the immunoglobulin Fc region is a hybrid of two or more domains selected from the group consisting of IgG, IgA, IgD, IgE, and IgM, said domains having different origins derived from immunoglobulin.

9. The FacVIIa complex of claim 7, wherein the immunoglobulin Fc region is a dimer or multimer of a single chain immunoglobulin composed of domains of same origin.

10. The FacVIIa complex of claim 7, wherein the immunoglobulin Fc region is an IgG4 Fc region.

11. The FacVIIa complex of claim 10, wherein the immunoglobulin Fc region is an aglycosylated human IgG4 Fc region.

12. The FacVIIa complex of claim 1, wherein the non-peptidyl polymer has a functional group selected from the group consisting of an aldehyde, a propion aldehyde, butyl aldehyde, maleimide, and a succinimide derivative.

13. The FacVIIa complex of claim 12, wherein the succinimide derivative is succinimidyl propionate, succinimidyl carboxymethyl, hydroxy succinimidyl, or succinimidyl carbonate.

14. The FacVIIa complex of claim 12, wherein the non-peptidyl polymer has a reactive aldehyde group as a functional group at both or three termini.

15. The FacVIIa complex of claim 12, wherein the non-peptidyl polymer has a reactive aldehyde group as a functional group at each terminus.

16. The FacVIIa complex of claim 15, wherein the non-peptidyl polymer is polyethylene glycol.

17. A pharmaceutical composition for blood coagulation, comprising the FacVIIa complex of claim 1.

18. A method for preparing the FacVIIa complex, comprising:
(1) linking a non-peptidyl polymer having an aldehyde, maleimide or succinimide derivative as a functional group at a terminal to an amine or thiol group of an immunoglobulin Fc region via a covalent bond to give a conjugate;
(2) isolating the non-peptidyl polymer-immunoglobulin Fc region conjugate from the reaction mixture of step (1);
(3) covalently linking FacVII to another end of the non-peptidyl polymer of the isolated conjugate to afford a FacVII complex in which the non-peptidyl polymer is linked at one end to the immunoglobulin Fc region and at another end to FacVII; and
(4) activating the FacVII complex of step (3) into a FacVIIa complex in which FacVIIa is linked to the immunoglobulin Fc region via the non-peptidyl polymer.

19. A method for preparing the FacVIIa complex, comprising:
(1) linking a non-peptidyl polymer having an aldehyde group at each terminus to the N-terminus of an immunoglobulin Fc via a covalent bond at a pH of 5.0 to 7.0 to give an immunoglobulin-non-peptidyl polymer conjugate;
(2) isolating the conjugate from the reaction mixture of step (1);
(3) covalently linking FacVII to another end of the non-peptidyl polymer of the conjugate to form a FacVII complex in which the non-peptidyl polymer is linked at one end to the immunoglobulin Fc region and at another end to FacVII; and
(4) activating the FacVII complex of step (3) into a FacVIIa complex in which FacVIIa is linked to the immunoglobulin Fc region via the non-peptidyl polymer.

20. The method of claim 18, wherein the non-peptidyl polymer has an aldehyde at each terminus and is covalently linked to an N-terminus of a light chain of FacVII.

21. The method of claim 18, wherein the FacVII is covalently bonded at an N-terminus to the non-peptidyl polymer.

22. The method of claim 18, wherein the FacVII complex is activated by on-column activation or in-solution activation.

23. The method of claim 18, wherein the FacVII and the FacVIIa are respectively native FacVII and native FacVIIa.

24. The method of claim 18, wherein the non-peptidyl polymer is polyethylene glycol.

25. A method of treating a blood coagulation-related disease, which comprises administering the FacVIIa complex of claim 1 to a subject in needs thereof.

26. A method of treating a blood coagulation-related disease, which comprises administering the pharmaceutical composition of claim 17 to a subject in needs thereof.

27. The method of claim 25, wherein the blood coagulation-related disease is hemophilia, bleeding, acute intracerebral hemorrhage, a wound or FacVII deficiency.

28. The method of claim 26, wherein the blood coagulation-related disease is hemophilia, bleeding, acute intracerebral hemorrhage, a wound or FacVII deficiency.

29. The method of claim 19, wherein the FacVII is covalently bonded at an N-terminus to the non-peptidyl polymer.

30. The method of claim 19, wherein the FacVII complex is activated by on-column activation or in-solution activation.

31. The method of claim 19, wherein the FacVII and the FacVIIa are respectively native FacVII and native FacVIIa.

32. The method of claim 19, wherein the non-peptidyl polymer is polyethylene glycol.
